(19) United States
(12) Patent Application Publication
(10) Pub. No.: US 2025/0099781 A1
(43) Pub. Date: Mar. 27, 2025

(54) Non-invasive Laser Based Therapy Device and Method For Treatment of Female Genitalia and Other Bodily Cavities

(71) Applicant: AEROLASE CORPORATION, Tarrytown, NY (US)

(72) Inventor: Pavel V. Efremkin, Sleepy Hollow, NY (US)

(73) Assignee: AEROLASE CORPORATION, Tarrytown, NY (US)

(21) Appl. No.: 18/972,933

(22) Filed: Dec. 7, 2024

Related U.S. Application Data
(63) Continuation-in-part of application No. 18/752,458, filed on Jun. 24, 2024.
(60) Provisional application No. 63/522,764, filed on Jun. 23, 2023.

(51) Int. Cl. A61N 5/067; A61N 1/04; A61N 1/30; A61N 5/06

(52) U.S. Cl. CPC A61N 5/067; A61N 1/0428; A61N 1/306; A61N 5/0624; A61N 2005/0606; A61N 2005/0611; A61N 2005/0626; A61N 2005/0659; A61N 2005/0666; A61N 2005/0668

(57) ABSTRACT

Apparatus and method for treatment of medical conditions associated with bodily cavities are disclosed. A laser generating device is provided producing 1064 nm laser wavelength beam having pulse duration between 0.1 and 3 milliseconds, and more specifically having 650 microsecond pulse duration. In the treatment of the medical conditions the bodily cavities are irradiated by this laser beam.

### CROSS REFERENCE TO RELATED APPLICATION

This application is Continuation-in-Part application of U.S. patent application Ser. No. 18/752,458 Jun. 24, 2024, which claims the benefit of U.S. Provisional Application Ser. No. 63/522,764 filed by the applicants on Jun. 23, 2023, both Applications are incorporated in their entirety herein by reference.

### FIELD OF THE DISCLOSED TECHNOLOGY

The present invention relates in general to the field of laser medical devices, and in particular it relates to a laser based female genitalia and vaginal and other bodily cavities therapy device.

### BACKGROUND OF THE DISCLOSED TECHNOLOGY

As women go through childbearing, hormonal changes and aging, they can experience changes in their genitalia including but not limited to external and internal sex organs, glands as well as surrounding skin areas. These changes can impact a woman's quality of life and may include vaginal laxity, stress urinary incontinence, lichen sclerosis, vaginal infections (Bacterial vaginosis (BV), Yeast infections, etc.), loss of vaginal lubrication (dryness), a decrease in erotic sensation, as well as changes in outside skin areas aesthetic appearance such as aging, dry, and sagging skin, hyperpigmentation, unwanted hair, etc.

Devices have been developed to treat some or all the above-noted changes through the use of laser energy, electromagnetic, radiofrequency or ultrasound. The field of nonsurgical female genitalia laser based therapy device is growing as there is a big demand to address such pervasive medical conditions that affect big female populations as well as there is growing demand for overall increase of quality-of-life attention.

Energy based devices currently used to address aging conditions in the vagina such as vaginal laxity, stress urinary incontinence, loss of vaginal lubrication (dryness) designed to create limited wound or trauma to the treated vaginal tissue areas inside the vagina to stimulate a healing response that may induce rebuilding of vaginal/and other bodily cavities tissue in the treated areas which may result in improvement is vaginal walls laxity as well as restoring the functionality of internal surfaces of vaginal tissue However, the mechanism of action currently used devices to creating wounds in vaginal and other bodily cavities tissue introduce risk of infections and/or uncontrolled injury which may result in scarring. Currently used fractionated ablative lasers create open wounds by using ablation to vaporize the internal vaginal walls tissue and create micro-channels of injury. Such open wounds create access for potential infections, and post treatment discomfort as well as it requires a treated patient to abstain from sexual intercourse for at least two weeks post treatment. RF (radiofrequency) devices create a surface injury to the vagina walls which carry a risk of serious injuries if operator deviates from proper protocol and most of RF treatments require manual and operator dependent application. Ultrasound technologies create substantial mechanical destructive injuries to vaginal and other bodily cavities tissue and require high precision in application to avoid potentially serious injuries. Also, RF and ultrasound technologies do not address infections which may already exist in vaginal and other bodily cavities areas and fractionated ablative lasers may only partially affect such infections because of their fractionated way of delivering energy in small less than 1 mm dimeter points. Also fractionated laser, RF and ultrasound technology cannot effectively address ageing related concerns in the external genitalia areas such as lichen sclerosis, hyperpigmentation, unwanted hair etc.

There is a clearly unmet need on the market for a safe non-invasive method of inducing collagen stimulation and vaginal and other bodily cavities tissue laser based therapy device without risk of infections from open wounds and/or unwanted injuries risk potentially associated with currently used technologies. It would be also highly advantageous if the same device could safely and effectively address ageing related concerns in the external genitalia areas such as lichen sclerosis, hyperpigmentation, unwanted hair etc. Most of the currently used devices and/or technologies typically address a very limited number of age-related conditions in the genitalia area. This requires medical practitioners specializing in female genital treatments to own and use multiple specific devices resulted in the increased cost of health care.

Laser energy devices with optimally selected parameters of wavelength, pulse duration, beam profile and energy density/fluence were proven to non-invasively and selectively affect certain skin or other soft tissue structures to deliver desired effect on such tissue restoration or laser based therapy device.

Thus, it has been long felt and unsolved need for a versatile, universal all in one laser-based apparatus which can effectively address a majority of the above listed internal and external female genitalia medical and aesthetic concerns in safe, non-invasive, nonsurgical manner. It has been also a need for a versatile, universal all in one laser-based apparatus and method for treatment of other bodily cavities, such as for example rectum in female and male patients.

It has been also an acute need for a laser based safe and non-invasive vaginal and other bodily cavities laser based therapy device apparatus which is simple in use and causes the increased collagen formation with the improved vaginal tightening.

### BRIEF DESCRIPTION OF THE INVENTION

Nd: YAG 1064 nm lasers proved to be capable of affecting skin or soft tissue structures in a selective and a non-invasive way. It is known that 1064 nm laser energy is absorbed in 3 main elements of human tissue: water, hemoglobin and melanin. These factors position 1064 nm laser as a potentially good and promising platform to develop a versatile laser device for female genital laser based therapy device. Long pulsed 1064 nm lasers with pulse duration range between 3 milliseconds (ms) and 50 ms are proved to deliver high energy fluences deep into the skin or soft tissue up to 5 mm depth. However, exposure to laser energy with such pulse duration can result in superficial burns, pain and other undesirable side effects. 1064 nm lasers with nanosecond or picosecond pulse duration are not capable of generating high energy laser fluences and their energy is absorbed superficially which makes them to be non-effective for the discussed applications. High power 1064 nm lasers with pulse durations between 0.1 ms and 2 ms allow for an optimum combination of high laser fluences sufficient to achieve a desired therapeutic effect but associated with minimum risk of surface thermal injury to the treated tissue. 1064 nm lasers with pulse duration range between 0.5 ms and 0.8 ms are the most optimal for the selected treatment. This is because such lasers are capable of delivering the highest laser energy safely and with maximum soft tissue penetration. The 0.65 ms (650 microsecond) lasers are in the middle point within the tolerances of this optimal range.

The 1064 nm laser with 650 microsecond pulse duration is proven to be a highly efficacious tool for the removal of unwanted pigmentation and/or hair. This is because of the favorable absorption of such pulses in melanin as well as the effective skin tightening qualities/characteristics due to laser energy absorption in water. This makes 650 microsecond pulse duration lasers as an optimal tool to address skin aging concerns in exterior female genitalia areas.

Skin treatment lasers for the removal of unwanted hair, pigmentation and skin tightening emit a laser beam from a handheld instrument/unit which applies laser energy directly to the skin. This method of delivery of laser energy cannot be effectively used to deliver laser energy to the internal body cavities such as vaginal and other bodily cavities areas or rectum. To resolve this issue the invention provides a special additional tool/device which effectively delivers the laser beam from the handheld laser instrument/unit into the closed/internal vaginal areas and/or other bodily cavities of female and male patients including rectum. Such an additional device is easily and reliably attachable to the handheld laser instrument/unit. Thus, the invention provides medical practitioner with easy to use a unitary instrument applicable for treatment of o both external and internal female genitalia areas and/or other bodily cavities of female and male patients including rectum.

Such internal vaginal and other bodily cavities laser based therapy device is optimally designed to deliver laser energy into internal vaginal areas in accordance with specified protocol determining the optimal and uniform exposure of internal vaginal areas to the laser energy. The device of the invention includes various sensors to control the effect of laser energy on the vaginal and other bodily cavities tissue (Including but not limited to surface temperature of treated area, etc.) and also includes sufficient safety features preventing delivery of laser energy if all of the safety protocol requirements are not satisfied.

Prior art laser devices for treatment of female genitalia typically provide fractional irradiation to particular spots which do not provide the full surface related coverage and create grid channels of FIG. 10B showing micro-channels of injury created by fractionated ablative lasers that create open wounds by using ablation to vaporize the internal vaginal and other bodily cavities walls tissue or FIG. 10C with other types of fractionated lasers creating the micro-channels thermal damage wounds. In the current invention, as illustrated in FIG. 10A laser energy is typically delivered to the treated area in the form of large approximately 6 mm round spots that do not ablate or otherwise injure the top surface of the internal vaginal and other bodily cavities wall but non-invasively penetrate through the vaginal and other bodily cavities wall thickness delivering laser energy for the desired therapeutic effect. For maximum therapeutic effect the coverage of the entire treated area must be complete with no untreated areas left. In the treatment method invention this is achieved by delivery of optimally overlapping laser spots to the treated area in a circular motion application manner. In addition, such application provides full coverage of internal vaginal and other bodily cavities area by means of delivery of optimally overlapping laser spots to the treated area in longitudinal application manor to provide full coverage of internal vaginal and other bodily cavities area longitudinally. It is also important to prevent laser firing into the same spot without proper time interruptions between laser pulses (which should be determined by taking into consideration the laser wavelength, fluence, pulse duration, spot size, overlapping area etc.) to allow the tissue to cool to prevent thermal injury. The vaginal laser device of the invention includes mechanical, software, and sensor solutions to manage and control optimal and safe delivery of laser energy to the genital areas. The optimal internal vaginal and other bodily cavities area coverage by overlapping laser spots according to the method of the invention is illustrated in FIG. 11.

The method of the invention comprises the steps of delivery optimally overlapping laser spots to the treated area in circular application motions as well as to provide full longitudinal coverage of internal vaginal and other bodily cavities area (circumvention and by overlapping laser spots-on treated area in longitudinal application manor to provide full coverage of internal vaginal and other bodily cavities area longitudinally). Such application prevents multiple laser firings to the same tissue spot without proper time interruptions between laser pulses (which should be determined taking into consideration the laser fluence, spot size, overlapping area etc.) to allow tissue to cool between pulses and to prevent thermal tissue injury. To achieve this goal the vaginal/and other bodily cavities laser device of this invention includes mechanical, software, and sensor arrangements to manage and control optimal and safe delivery of laser energy to the genital areas. Optimally predetermined numbers of circular overlapping motions of a laser beam being delivered to the skin tissue back and forth along the internal vaginal and other bodily cavities area creates an enhanced protocol for achieving desired medical and laser-based therapy device effects as well killing infection. The optimally predetermined numbers of circular overlapping motions of a laser beam as well as the longitudinal step dimension and the number of longitudinal movement cycles are optimally determined using the predetermined algorithm of the software and inputs from the sensors that reads the physical characteristics of the laser treated tissue.

Full coverage of internal vaginal and other bodily cavities area and delivery of sufficient amount of laser energy allows for treatment of vaginal/and other bodily cavities infections (Bacterial vaginosis (BV), Yeast infections, etc.) as well as increasing efficacy of collagen stimulation and rejuvenation of vaginal and other bodily cavities tissue.

The vaginal and other bodily cavities laser device must be designed to address health risks of both treated patient and medical practitioner delivering the treatment. To protect a patient vaginal and other bodily cavities laser device of the invention is equipped with a single use hygienic disposable shield 70 (See FIGS. 4, 6, 7 and 8) which prevents transmission of infectious diseases between individual patients.

The 650 microsecond 1064 nm laser equipped with a detachable vaginal and other bodily cavities laser delivery instrument allows for an optimum universal all in one medical laser-based apparatus to treat most of aged related female genital area concerns. Without detachable vaginal and other bodily cavities laser delivery instruments such lasers are an effective tool for unwanted pigmentation and hair removal, external skin areas rejuvenation and tightening, lichen sclerosis and other medical conditions. With the detachable vaginal and other bodily cavities laser delivery instrument such laser apparatus treats vaginal laxity, stress urinary incontinence, vaginal infections (Bacterial vaginosis (BV), Yeast infections, etc.), loss of vaginal lubrication (dryness), a decrease in erotic sensation, and other conditions.

The 650 microsecond 1064 nm laser equipped with a detachable vaginal or rectum laser delivery instrument can be effectively used for treatment of hemorrhoids or swollen veins in the anus and lower rectum. 1064 nm laser energy is effectively absorbed in oxyhemoglobin which makes it an effective tool to treat varicose veins, and other vascular malformations. Hemorrhoids located in the rectum and anus area have the same physiology. The device of the invention provides effective delivery of 1064 nm laser energy to the affected areas inside the rectum and 650 microsecond pulse duration allows for safe and comfortable treatment with no side effects like burns or pain.

### SUMMARY OF THE INVENTION

One aspect of the invention provides 650 microsecond 1064 nm laser formed with a detachable vaginal and other bodily cavities laser delivery instrument/arrangement/device that allows for an optimum universal all in one medical laser-based apparatus to treat most of aged related female genital area concerns and hemorrhoids. It should be noted however that without the detachable vaginal and other bodily cavities laser delivery instrument such laser is an effective tool for unwanted pigmentation and hair removal, skin areas rejuvenation and tightening, lichen sclerosis and other medical conditions. With the detachable vaginal and other bodily cavities laser delivery instrument such laser apparatus effectively treats vaginal laxity, stress urinary incontinence, vaginal infections (Bacterial vaginosis (BV), Yeast infections, etc.), loss of vaginal lubrication (dryness), a decrease in erotic sensation, and other conditions.

Another aspect of the invention provides vaginal and other bodily cavities laser based therapy device that when attached to a laser energy emitting source/handpiece translates emitted laser energy in such a way that allows safe irradiation of internal walls of vagina or other similar internal body areas in a uniform, controllable way.

Another aspect of the invention provides a vaginal and other bodily cavities rejuvenation device with disposable safety shield with the support formation. The device comprises an enclosure tightly surrounding and covering the entrance into vaginal and other bodily cavities area to protect female patients and medical practitioner from the potential bacteria, viruses, gasses or fluids that can be produced and spread through the air during the laser treatment. The support formation is formed having soft edges to provide maximum tight contact with the skin of a patient.

A nozzle is provided facilitating connection to medical vacuum, which allows for creation and vacuum suction enhancing attachment of the support formation to the skin of the patient. The nozzle also is used for the removal of unwanted substances with further filtering such substances into the disposable filter.

The nozzle associated with the support formation also includes a back flow valve for keeping the unhealthy substances within the support part cavity in case of interruption of the vacuum suction removal.

The vacuum suction is provided by an air-suction, which also used to cool laser generator, wherein the support formation connected to the laser air-vacuum cooling system through disposable hose includes a single use air-filter for catching and storing the unwanted air-borne substances produced during vaginal laser treatment.

The vaginal and other bodily cavities rejuvenation device of the invention is optimally designed to deliver laser energy into the internal vaginal and other bodily cavities areas in accordance with specified protocol determining the optimal and uniform exposure of internal vaginal and other bodily cavities areas to the laser energy. The apparatus of the invention includes various sensors to control the effect of laser energy on the vaginal and other bodily cavities tissue (Including but not limited surface temperature of treated area, etc.) and also includes efficient safety features preventing delivery of laser energy when the safety protocol requirements are not satisfied.

The algorithm controlling optimal numbers of circular overlapping motions of a laser beam being delivered to the skin tissue back and forth along the internal vaginal and other bodily cavities area can be optimally adjusted to achieve desired medical and rejuvenation effects based on inputs from the sensors that reads the physical characteristics of the laser treated tissue.

### BRIEF DESCRIPTION OF THE DRAWING

The invention is further illustrated by way of example and not limited to the figures of the accompanying drawings, in which reference numerals indicate corresponding, analogous or similar elements. Further, where considered appropriate, reference numerals may be repeated among the figures to indicate corresponding or analogous elements.

FIG. 1 is a view showing a laser apparatus of the invention in the assembled condition provided for treatment of a vaginal and other bodily cavities.

FIG. 2 is a partial view of the apparatus of the invention.

FIG. 3 is a sectional view of the laser apparatus of the invention illustrated in FIG. 1.

FIG. 4 is a rear view of the hygienic shield.

FIG. 5 shows absorption chart for 1064 nm wavelength laser light irradiation in three main building blocks of the skin tissue: water, oxyhemoglobin, and melanin, the chromophores non-invasively and controllably affected and utilized by the laser energy of the vaginal and other bodily cavities rejuvenation laser device of the invention.

FIG. 6 is an enlarged partial view illustrating operation of a laser reflecting/dispensing arrangement of the invention.

FIGS. 7 and 7B are schematic diagrams illustrating various applications of the laser device of the invention to the treatment of the vaginal and other bodily cavities.

FIGS. 8A and 8B are sectional views showing various embodiments of the protective shield.

FIG. 9 is a schematic diagram illustrating suctional and cooling functions of the apparatus of the invention.

FIG. 10A is a diagram illustrating a non-ablative laser treatment.

FIG. 10B is a diagram illustrating a fractional ablative laser treatment.

FIG. 10C is a diagram illustrating a non-ablative fractional laser treatment.

FIG. 11 is a schematic diagram illustrating overlapping principal concepts of the invention.

FIG. 12 is a schematic diagram illustrating a further embodiment of the invention.

### DETAILED DESCRIPTION OF THE DISCLOSED TECHNOLOGY

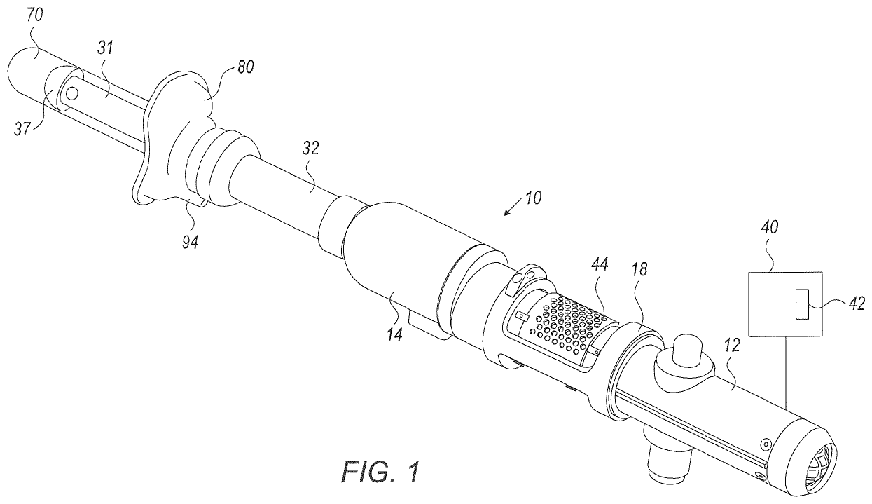

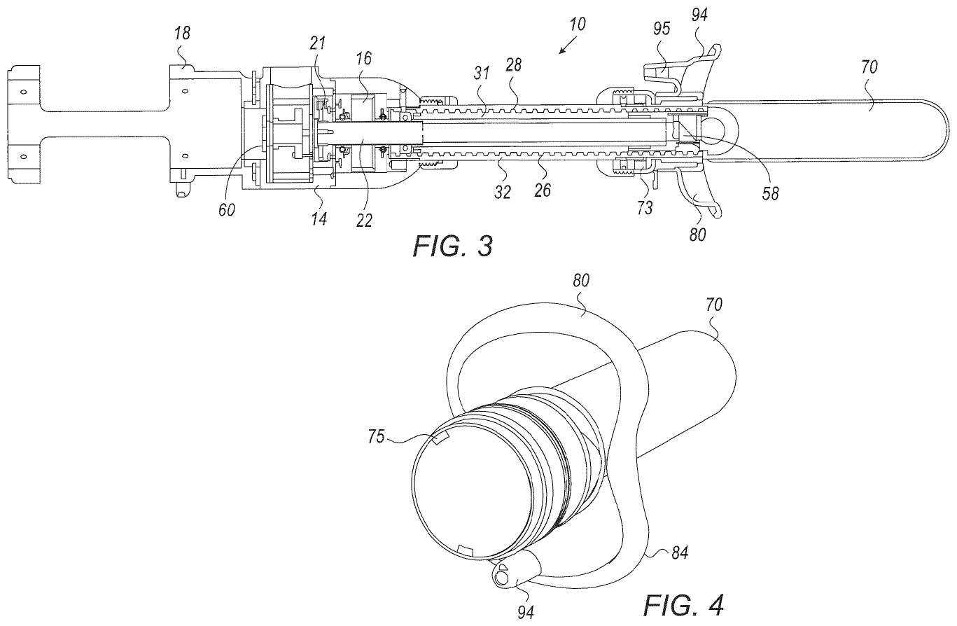

Referring now to FIGS. 1 and 3 best illustrating a laser device/apparatus 10 of the invention applicable to treatment of the vaginal and other bodily cavities in the assembled condition and attached to laser source/laser handpiece. Among essential elements of the device/apparatus 10 are: a rotary actuator or an electrical motor 16, a substantially hollow external housing 32 that houses a mechanical arrangement that translates rotational movement of the hollow shaft of the rotary or actuator electrical motor 16 into longitudinal movement of a tubular laser energy dispensing arrangement 20 that houses a laser reflecting member or an optical prism 58 that reflects the laser beam emitted by the laser handpiece at 90-degree angle for application to the internal walls of vagina, a laser handpiece module 12 that emits laser energy beam along its longitudinal axis, and a connecting brace 18 that allows to optionally connect the rotary actuator 16 part of the device/apparatus 10 and a laser handpiece 12. A hygienic disposable shield assembly 70 protects patients from contact with mechanically moving parts of laser irradiation dispensing arrangement 20 and protects arrangement 20 from body liquids and other condition or elements. Applications of the laser handpiece module 12 as a part of the apparatus of the invention is mainly directed to the treatment of the internal parts of the female genitals and are discussed in full detail in the present application. However, the laser handpiece module 12 taken independently can be used by a practitioner for applying laser pulses directly to the skin of a patient including applications to the treatment of various external skin tissue conditions.

As best illustrated in FIG. 3 in the preferred embodiment of the invention the rotary actuator in the form of an electrical step motor 16 is provided within case 14. An output shaft 22 of the electrical motor is connected to a rear end of the connecting element for mutual rotation. The incremental rotation of the electrical motor 16 translates into the corresponding rotation of the telescopic assembly 30. Although the rotary actuator in the form of the electrical step motor 16 has been discussed in the application, other rotary actuators, such as a multi-phase AC motor, a brushless DC motor or their functional equivalents to these actuators may also be used in the invention.

Brace 18 provides a fixed optically aligned connection between case 14 of the electrical motor and the laser handpiece module 12. A central region or a core 21 of the motor is formed with an opening accommodating a rotatable hollow shaft 22 allowing a laser beam generated in the laser handpiece module 12 to pass through the entire apparatus 10 to an optical prism 58 that reflects the laser beam emitted by the laser handpiece at 90 degrees for application to the internal walls of vagina.

The laser handpiece module 12 is configured to include at least a laser emitter and a lens or optical window removably associated with each other. The lens extends into the rear opening of the case 14 and is disposed substantially perpendicular to the optical axis of the laser. Design and operation of the laser handpiece module 12 is described in greater detail by the inventor's own U.S. Pat. Nos. 5,868,731; 6,251,102 and 6,813,289 which the present application incorporates by reference in their entirety.

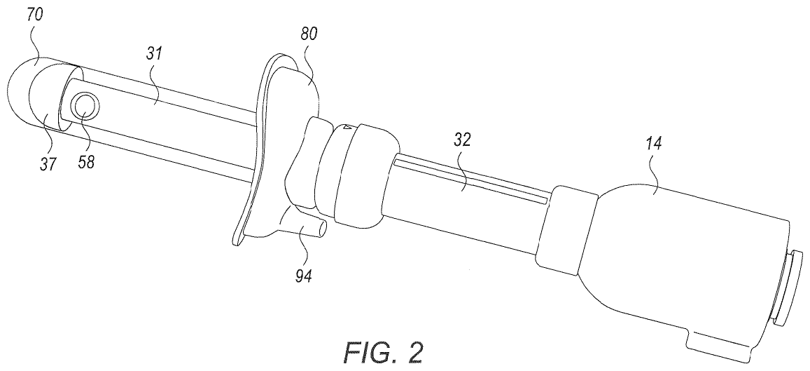

FIG. 2 shows the device/apparatus 10 of the invention in the assembled condition separately from the laser handpiece.

According to the preferred embodiment a laser device 10 which can be used for treatment of the vaginal and other bodily cavities is best illustrated in FIGS. 1-3 and 6. The device 10 comprises a substantially hollow external housing 32 with a tubular receiving element 31 accommodating the shaft 22 positioned within the hollow housing interior. External housing 32 is connected to the electrical motor case 14. The receiving element 31 is formed having hollow interior and exterior regions. The shaft 22 passes through the interior region, with the external threads 26 to be disposed at the exterior region. The internal threads 28 are formed at an inner surface of the hollow external housing 32.

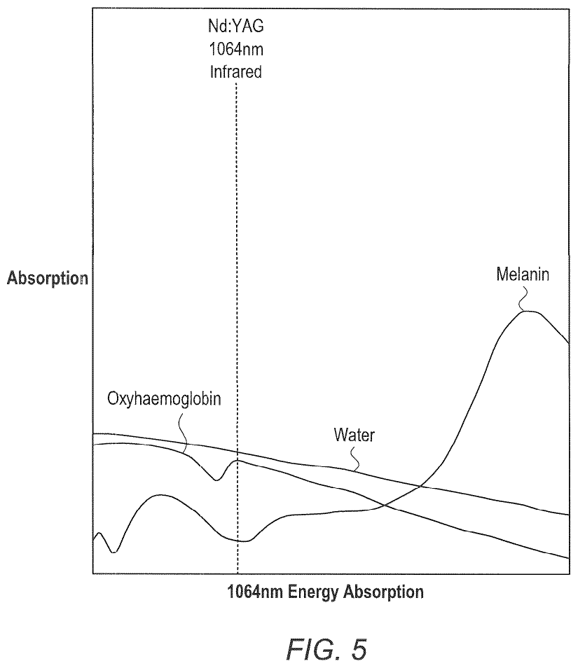

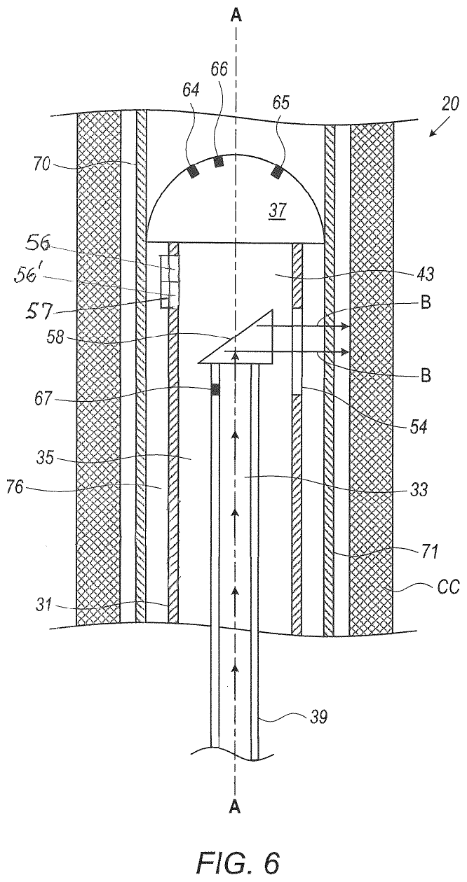

By a special mechanical setting the rotational motion of the hollow shaft 22 and the laser dispensing arrangement 20 (see FIG. 6) provided at the distal end thereof, is converted into their own linear motion. In the preferred embodiment of the invention, as best illustrated in FIG. 3, screw threads are provided to convert rotational motion of the motor 16 and the hollow shaft 22 into the linear motion of the operational telescopic assembly including the laser beam reflecting arrangement or prism 58 within the housing. External threads 26 are wrapped around the receiving element 31 in the form of a helix, whereas the internal threads 28 are wrapped around the interior region of the hollow housing 32. The external threads of the receiving element engage the internal threads of the housing. Thus, laser dispensing arrangement 20 (see FIG. 6) converts the laser energy passing through the interior and along a longitudinal axis of the apparatus 10 to be ultimately directed at about 90-degree angle to the longitudinal axis, to irradiate the interior of the vaginal and other bodily cavities. The mechanical advantage of a screw thread arrangement depends on its lead, which is the linear distance the receiving element and the whole telescoping assembly travels within the housing in one revolution of the motor and the connecting element. On the other hand, it should be clear that many other technical variations for the conversion of the rotational motion into the linear motion of the elements are within the scope of the invention.

During the incremental motion of the motor 16, the shaft incrementally rotates to a certain degree. For example, 10 degrees rotation of the shaft can be accomplished during each incremental motion of the step motor. Incremental rotational motion of the shaft is energized by the motor. In this motion the connecting element rotates along the longitudinal axis A-A of the assembly. Thus, the whole operational assembly powered by the motor rotates about the longitudinal axis A-A. During this rotation the receiving element can slide on the connecting element due to the telescopic arrangement/connection therebetween. Therefore, the assembly undergoes a combined motion which provides for rotation of the assembly and slidable/telescopic movement between the receiving and connecting elements. In the preferred embodiment the receiving element slides over the connecting element.

A shutter 60 is provided in the therapy device of the invention to either intercept or block the light beam produced by the laser source or to permit passage of the beam to the reflecting member 58. The shutter operates between either open or closed positions. In the working conditions the shutter is opened, and the laser beam is allowed to extend through the entire assembly of the apparatus 10.

The shutter 60 remains closed when no flash is detected, preventing undesirable/uncontrollable lasing of the tissue. As an optional feature, warning sound or other signal can be emitted by the control unit 42 alerting the user about lack of the progress in the procedure. The control unit 42 will close the shutter if no flash is detected for longer than a predetermined time interval, 5 seconds for example. This arrangement provides well-controlled safety operation.

The laser source may be selectively operated to produce a coherent light beam which exits the front of the housing. The shutter 60 may comprise a generally planar blade made of or coated with a dielectric material which can withstand the impingement thereon by the laser beam and which blocks the beam when the shutter is in its first/closed position. In one embodiment, the shutter blade is made of a dielectric material coated with a reflective material. In an alternate embodiment the shutter blade is a mirror. The coating is selected so that the blade can act as an efficient mirror for light having the wavelength of the light/laser beam. The shape of the shutter blade may be planar or non-planar, such as convex.

The following are essential elements of the system. The shutter blocks the port-entrance for the laser beam/energy. When the device is not operational or not in the working condition, the shutter blocks the port-entrance preventing passage of the laser beam into the interior of the device. In this manner, the laser beam cannot reach the reflecting member 58 though the interior of the telescopic assembly and is prevented from being delivered to a tissue of the patient. As a safety feature, the shutter is also activated by control unit 42 when any adversarial situation arises. For example, when any problem with the device occurs, such as the motor is stocked, or a surge of the power occurs, the shutter 60 is automatically activated preventing the laser beam from passage into the interior of the assembly and ultimately to the vaginal/and other bodily cavities tissue of a patient.

In the invention the shutter is independent from the laser. The elements of the apparatus of the invention are mechanically and not electronically interconnected. The electric motor 20 is activated by the laser light itself. As the laser emitter is activated and pulsing occurs, the photo sensor generates a signal to initiate rotation of the motor 16 and the telescopic assembly 30 at a preset/predetermined rotational angle. The rotation of the motor 16 occurs only when the laser emitter is energized, and pulses are generated. This is an essential safety feature of the device. The elements of the device/apparatus are not connected electronically to the source of laser energy or the emitter. However, when the photo sensor reads the flash from the laser the shatter 60 is moved to an open, working position. Then the reflecting element/mirror 58 is moved within the housing and the receiving element in accordance with the required protocol.

Optional features of the laser-based therapy device n of the invention may include electrodes for vaginal and other bodily cavities muscle simulation and/or contraction to stimulate muscle strength and tone improvement. It may also include a vibratory device for sensitivity stimulation. Application of a gel may be used to optimize the applicable reflection coefficient. Openings in the external surfaces can be formed to release the required or excessive amounts of gel. Depth marks can be also provided at the exterior of the shield 70. Use of temperature sensors, electrodes for RF delivery, or outlets for sonic or ultrasound waves are also contemplated. An automatic recording of the treatment parameters and their transmission to a database for centralized information processing can also be provided.

It will be discussed later in a substantial detail that laser-based therapy device laser device of the invention for treatment of the vaginal and other bodily cavities is used in combination with a removable and disposable protective shield 70 provided at an exterior of the receiving element 31.

Turning now to FIG. 6 which is an enlarged detailed partial view illustrating structure and operation of a laser energy dispensing arrangement 20 of the laser device of the invention. As illustrated, an operational head or the laser reflecting unit 58 is provided at the distal end of the connecting element 39. The reflecting member/unit 58 is disposed at the optical axis A-A of the apparatus for intercepting and reflecting the emitted radiation of the laser beam that passes through a channel 33 in the hollow interior of the connecting element 39 The reflecting member 58 can be made from non-metallic materials, such as glass and may be coated with a reflective material to further reduce the absorption of laser energy while reflecting the energy to the body site. In a preferred conceptual embodiment, the reflecting member 58 is a mirror that reflects light at 90° (5°-175°).

At one window 54, is formed within the wall of the hollow body of the receiving element 31. The window 54 can be formed having either a round rectangular configuration. The reflecting member/prism 58 is positioned in the hollow interior 43 in the vicinity of the window 54. The material of window 54 is transparent to laser light with high durability (energy threshold).

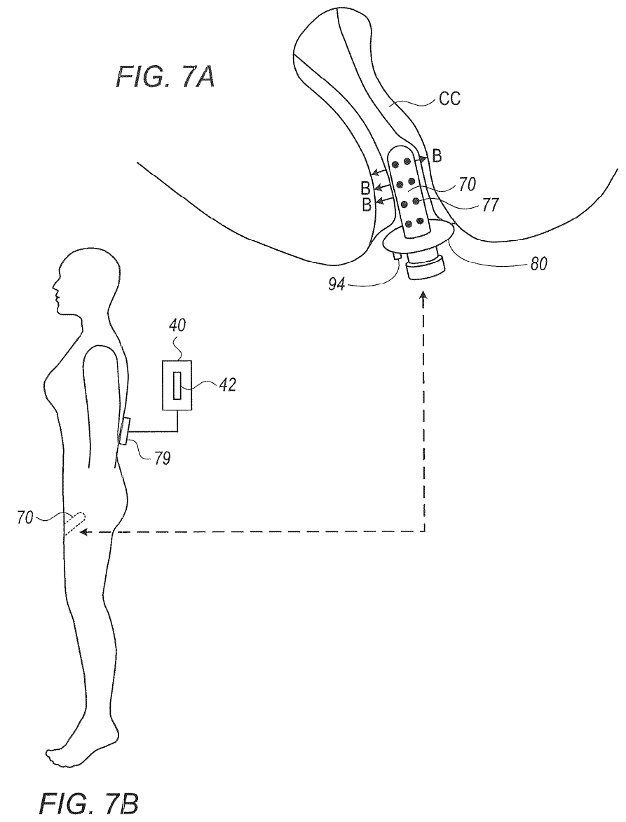

It is further illustrated in FIGS. 6 and 7 that the laser radiation, emitted by the laser energy source or the emitter of the handpiece 12, is transmitted through the channel 33 in the hollow interior of the connecting element 39 and is intercepted by the reflecting member 58. The radiation is reflected transversely of, and substantially around the longitudinal axis A-A by the reflecting member 58. The reflected radiation BB (see FIG. 6) passing through a laser radiation transparent wall 71 of the shield 70 is thus laterally directed circumferentially to the target vaginal and other bodily cavities tissue CC (see also FIG. 7A) at the bodily cavities cavity site. This motion can be gradual/incremental with 10 degrees intervals, for example. Thus, the entire laser dispensing assembly powered by the motor 16 rotates about the optical/longitudinal axis A-A. During the rotation the receiving element 31 is capable of sliding on the connecting element 39. Therefore, the laser dispensing assembly 30 undergoes a combinational motion which consists of the incremental rotation of the entire assembly and the slidable movement.

In view of the above, the reflecting member 58 also involves two combined motions. According to the first motion the reflecting member 58, as a part of the receiving element 31 is rotated in a circular fashion within the shield interior 76. According to the second motion the reflecting member 58 moves linearly back and forth within the shield 70 in the longitudinal direction along the axis A-A. Thus, the reflecting member 58 makes rotational and linear passages covering the whole interior length of the shield 70 and target vaginal and other bodily cavities tissue CC at the applicable bodily cavity site.

Visual observation of the cavity interior by a doctor is highly desirable for proper treatment. As previously discussed, the apparatus of the invention includes window 54 for laser irradiation. In an alternate embodiment, as also illustrated in FIG. 6, at least two supplemental windows 56 and 56′ are provided to accommodate an imaging device 57. This imaging device can be a video or photo camera, enabling both real-time observation and recording of the interior of the bodily cavity.

The supplemental windows 56 and 56′ are typically disposed to allow a doctor to observe and analyze the targeted tissue before a laser beam passing through the window 54 is applied during the treatment. This allows to analyze the tissue and concurrently treat the patient's tissue by a laser beam passing through the laser window 54 in both in and out movement of laser rotating member.

Imaging device 57 typically consists of two parts each associated with respective windows 56 and 56′. The first part provides a doctor with observation of a lesion resulting in photographs and video files. A second part enables a doctor to observe internal walls of a bodily cavity and to analyze lesions in different wavelengths so as to determine a degree of vascularity, pigmentation or any other tissue characteristics. It also enables a doctor to detect a presence of bacteria.

When the imaging device 57 is used in manual mode, the practitioner, through operation of the control unit 40 and by means of the rotational motion of the entire dispensing assembly powered by the motor 16 and rotatable about the optical/longitudinal axis A-A, obtains a visual image of the cavity interior for various purposes, including locating and observing lesions.

By utilizing an image recognition function associated with the control unit 40, it is possible to scan the internal area of the bodily cavity and store the generated image, including location of the lesions, into the control unit 42 memory.

The imaging device 57, as part of the receiving element 31, is capable of 360-degree rotation. As a result, images of the lesions, including their coordinates, are generated and stored in the memory of the control unit 42. During the required treatment, laser energy is applied directly to the lesion based on the generated map or image. This generated image is stored in memory and can be retrieved as needed during the procedure. Consequently, erbium 2040 laser radiation can be emitted while the device follows the map or image stored in the control unit's memory. Thus, the invention provides an automated device for lesion treatment.

In an alternate embodiment multiple electrodes can be provided in the apparatus for RF treatment. Such multiple electrodes enable a doctor/practitioner to coagulate lesions of different sizes and configuration during RF treatment.

Medical conditions also known as cervical erosion, vaginal atrophy or vaginal ulceration have often resulted in heavily vascular lesions situated within the vaginal cavity. To ensure proper treatment, lesions of this type can be either obliterated or coagulated. The apparatus of the invention is designed to effectively coagulate vascular lesions, making it suitable for addressing the medical condition in question. In particular, the Erbium 2940 laser has demonstrated efficacy in the treatment of such lesions. Although the application of the apparatus and method described in this invention primarily pertains to the treatment of female genitals. However, it should be noted that the invention is also applicable to the ablation or irradiation of lesions located in other areas of both female and male patients, such as for example the rectum or other bodily cavities.

A semi-spherical formation 37 is provided at the distal region of the receiving element 31. The exterior diameter of the formation 37 is practically identical to the outer diameter of the external housing 32 allowing a tight contact. This is essential when the exterior of the device 10 is disinfected to protect internal mechanisms of the device 10 from liquid contamination. When the receiving element 31 is inserted into the external housing 32, the semi-spherical formation 37 serves as a barrier preventing penetration of moisture, etc. into the interior of the device 10.

Because of the medical reasons and a potential number of women being treated, it is advantageous to have a low-cost, disposable vaginal and other bodily cavities shield obviating the need for cleaning and sterilization of the instrument after use. This is particularly true considering the highly personal and sensitive nature of vaginal and other bodily cavities treatment and addressing the increase in the occurrence of sexually transmitted diseases. As used herein, “disposable shield” includes a device that is disposed of after use and is not intended for reuse. The disposable shield can be formed from plastic or another lightweight and inexpensive material penetrable to laser radiation.

As best illustrated in FIGS. 4, 7 and 8 the protective shield 70 is formed having a tubular shaped body extending between the front 72 and rear 74 ends. The rear end 74 is open and provided for insertion into or connection the external housing 32. A rounded front end 72 of the shield is smooth and is adapted to gently slide past body vaginal and other bodily cavities tissue in the vaginal/and other bodily cavities with a minimum of trauma to the vaginal and other bodily cavities tissue. At least the rounded front end can be coated with a non-stick or release coating to provide easy release from vaginal and other bodily cavities tissue.

A support formation 80 of the shield 70 is provided to facilitate optimal and consistent position of the device in the vaginal and other bodily cavities area. The support formation 80 is shaped as an enclosure which tightly surrounds and covers the entrance into vaginal and other bodily cavities area to protect a patient and a doctor from the potential bacteria, viruses, gases or fluids that can be produced and spread through the air during the laser treatment. The support part has soft outer edge 84 to assure maximum tight contact with the skin of a patient.

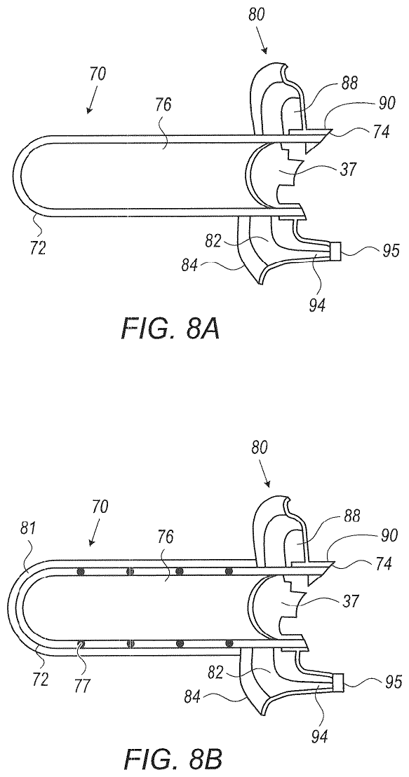

As best illustrated in FIGS. 4 and 8 the support or a stopping formation 80 having an inverted mushroom shape, is provided at an exterior of the rear end 74 of the disposable shield 70. A circumferential inner cavity 82 facing the front end of the shield is formed within the support. The cavity extends between an outer area defined by an outer edge 84 of the stopper and an inner area 88 associated with the tubular connector 90. The shape of the inner area 88 is adapted to accommodate an outer area of the female genitals.

An outside diameter of the tubular shaped shield 70 closely corresponds to a specific anatomy of the treated vagina. Thus, the treatment method of the invention includes a step of selection of the size of the shield including the length and/or diameter of the tubular shaped shield 70 to accommodate a specific virginal anatomy of a patient.

The material of the shield 70 is transparent to the laser radiation, so that the laser beam penetrates through the wall of the shield without any distortion. The material of the shield 70 should be also bio-compatible with the vaginal and other bodily cavities tissue.

A vacuum port or a nozzle 94 is provided for connection/communication of the cavity 82 within atmosphere. An essential function of the nozzle 94 is to generate a vacuum for evacuation of debris and/or gases developed during the treatment of the exterior female genitalia region. The entrance into the vaginal and other bodily cavities is shielded by the circumferential inner cavity 82. Further, the gases developed within the internal vaginal and other bodily cavities during the treatment upon entering the cavity 82 are evacuated to atmosphere through the nozzle 94.

As best illustrated in FIGS. 4, 8A, 8B and 9 the nozzle 94 is formed in the inverted mushroom-shaped support formation 80 to allow connection to a medical vacuum facilitating creation and vacuum suction. The support formation 80 attaches the device to the skin even closer to the patient as well as facilitates the removal of the unwanted substances with further filtering it into the disposable filter. The nozzle 94 also includes a back flow check valve 95 which keeps the unhealthy substances within the support cavity in case of interruption of the vacuum suction removal.

While the nozzle 94 is in use, the vacuum suction can be provided by a separately standing medical vacuum with disposable filter or by a suction mechanism of air-cooled medical laser apparatus. Modern medical laser devices may use air-suction as a means to cool laser generator. This allows also to have secondary use of this feature to replace a separately standing medical vacuum device. The support formation 80 can be connected to the laser air-vacuum with such disposable tube to include a single use air-filter to catch and store the unwanted air-borne substances which may be produced during vaginal and other bodily cavities laser treatment.

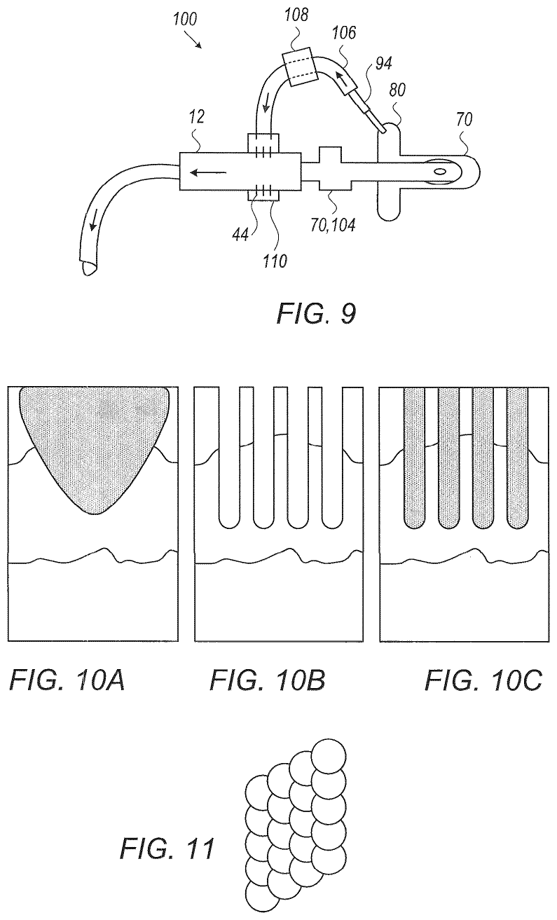

As illustrated in FIG. 9 a disposable filter 108 incorporated into the hose 106 is connected to the handpiece 12 interior and to air cooling system of the regular air-cooled laser (standard AEROLASE devise) which draws air through the handpiece interior.

The suction created by the air-cooling system of the air-cooled laser draws the air through the handpiece interior. The disposable air filter 108 facilitates filtering debris, wherein the suction/movement of the air is enhanced by the unique design of the invention. A distal end of the hose 106 is connected to the nozzle 94 of the support formation 80 and a proximal end is connected to an input port or a connector 110 provided at the handpiece exterior of the conventional laser device.

As best illustrated in FIG. 9, multiple apertures 44 are provided in the body of the laser handpiece 12 for air to be drawn into the handpiece interior. Since the port or a connector 110 is provided at the apertures 44 region for the connection between the distal end of the hose and the input area of the laser handpiece. The vacuum suction generated within the handpiece 12 interior is extended to the inner area of the hose 106. Such suction draws debris generated at the exterior of the female genitalia to be accumulated in the filter 108. Upon passing through the filter the cleaned air continues its movement into the interior of the laser handpiece 12 for its cooling. Thus, in the apparatus of the invention air provided for cooling the regular laser disposed in the handpiece 12 is also utilized to form the suction directed to remove debris produced at the external genitalia treatment area and accumulated in the disposable filter 108. Upon passage through the filter the purified air continues its movement through the hose 106 into the interior of the laser handpiece 12 for cooling.

In the method of the invention while the disposable safety shield 70 with the support formation 80 is utilized, an enclosure tightly surrounding and covering an entrance into an internal vaginal and other bodily cavities cavity is formed. Thus, the support formation 80 protects female patients and medical practitioners from the potential bacteria, viruses, gasses or fluids produced and spread through the air during the laser treatment.

Further, in the treatment method of the invention the support formation 80 including the nozzle 94 connected to medical vacuum are participated in the development of the vacuum suction which enhances engagement/connection between the support formation and the exterior skin of female genitalia to facilitate the removal of the unwanted substances debris with further filtering/removal of biological contaminants into the disposable filter 108. Still further, the nozzle 94 provided with a back flow check valve 95 prevents the discharge the unhealthy substances from the support internal cavity in case of interruption in the vacuum suction gas/biological contaminants removal.

In the method of the invention a vacuum based suction is generated based on air-suction which also used to cool laser generator with the support formation 80 connected to the laser air-vacuum cooling system through disposable hose 106 provided with a disposable air-filter 108 adapted to catch and retain/store the unwanted air-borne and other substances which may be produced during the vaginal and other bodily cavities laser treatment.

As a further aspect of the method, the nozzle 94 provides communication of the cavity 82 within atmosphere. In this manner the nozzle 94 is used to generate a vacuum for evacuation of the gases developed during the treatment at the exterior of the shield by suction. Thus, in the treatment method of the invention the shield, as a part of the device, is inserted into the vaginal and other bodily cavities, upon reaching a predetermined depth, it is stopped or prevented by the formation 80 from penetration dipper into the vaginal and other bodily cavities cavity. The entrance into the vaginal and other bodily cavities is shielded by the circumferential inner cavity 82, because some pressure should be applied to put it in. The gases developed within the vaginal and other bodily cavities during the treatment upon entering/penetration into the cavity 82 are evacuated to atmosphere through the nozzle 94.

Although in the preferred embodiment of the invention the disposable shield is used, utilization of multiuse shields which can be sterilized are also contemplated.

Apparatus 10 further includes a control unit 40 (best illustrated in FIGS. 1 and 7B) that controls many operational characteristics of the invention including the amount (including duration) of the laser light that is applied to the treated area. The control unit 40 can be provided to regulate the laser energy/power source for the optimum output level and other characteristics of the laser light (such as wavelength, pulse duration, pulse shape, repetition rate etc.) based on type and characteristics of the targeted vaginal and other bodily cavities tissue. Characteristics of the control unit 40 may be adjusted by the operator manually or automatically based on the signals and data received from the sensors 36 and 38 installed within the interior of the device.

Control unit 40 houses a programmable logic controller or microchip 42 to provide power and to control operation of various units of the invention. The control unit 40 also preferably incorporates control systems for actuating, adjusting and providing system information concerning laser power characteristics, axial and rotational movement of the laser reflecting arrangement 58 which displays reading of sensors located at the distal region of the receiving element. The control unit 40 also controls a block providing information concerning operating conditions and feedback from the vaginal and other bodily cavities treatment site to the operator. By means of a computer or microchip 42 the control unit 40 utilizes inputs received from multiple sensors 36, 38 located at the distal region of the receiving element and/or other critical regions of the device assembly to continuously updated output to an operator, including such operating parameters as laser parameters delivered to the treatment site, temperature at the treatment site, advance rate and the like.

Sensors 64, 65, 66 may emit and receive various types of signals (optical, electromagnetic, acoustical, capacitance measuring) that change parameters depending on the characteristics of the vaginal and other bodily cavities tissue, so as to allow the control unit 40 to calculate and generate proper signals controlling operation of the laser source or emitter.

Detectors/Sensors 64, 65, 66 are able to recognize (determine) the physical and chemical composition of the vaginal and other bodily cavities tissue. A computer or microchip 42 associated with control unit 40 receives and analyzes information/data obtained by the sensors and generates signals to adjust parameters of the power source to optimize the treatment and/or to produce other desired effect on targeted soft vaginal and other bodily cavities tissue.

According to one embodiment of the invention, sensors 64, 65, 66 can detect the level of water/moisture content, etc., within the targeted vaginal and other bodily cavities tissue. As shield/sleeve 70 and the laser reflecting arrangement 58 pass through various zones/sections/areas of the vaginal and other bodily cavities cavity, optimal levels of laser radiation can be achieved for each zone of treatment. For example, a lower level of radiation and/or higher repetition rate can be provided. On the other hand, higher levels of radiation and/or slower repetition rate will be generated and directed to the areas of treatment. Laser power sources may also be able to generate different wavelengths or pulse duration irradiation optimized for the most optimal effect on the targeted vaginal and other bodily cavities tissue.

Sensors 64, 65, 66 may emit and receive various types of signals (optical, electromagnetic, acoustical, capacitance measuring) that will change parameters depending on the composition or other physical properties of the treatment site and/or vaginal and other bodily cavities tissue surrounding the site, so as to allow the control unit 40 to calculate and generate proper signals controlling operation of the device of the invention.

Utilization of a laser based vaginal and other bodily cavities laser based therapy device of the invention is accompanied by automatic target feedback, thermal feedback for example, to precisely control the dosimetry of the laser irradiation. This is needed to prevent damage to the surrounding vaginal and other bodily cavities tissue. An output of the non-contact thermal detector 66 can be used to adjust the output of the laser power source to maintain selected characteristics including temperature at the treatment site.

Absorption of laser energy by the vaginal and other bodily cavities tissue may result in the temperature elevation of the surrounding vaginal and other bodily cavities tissue. In the invention, this occurs controllably without causing irreversible thermal damage to the surrounding tissues. The control unit 40 adjusts the energy to maintain a pre-selected target temperature at the site/spot. In one embodiment of the invention, to maximize patient safety, an optional continuous or pulsed cooling device can be provided to deliver a coolant to the treatment site during or after the laser rejuvenation procedure.

To further control the vaginal and other bodily cavities rejuvenation process, the condition of the vaginal and other bodily cavities tissue surrounding the treatment site is monitored by a detecting arrangement or detector 67 adapted to detect irradiation reflected from such vaginal and other bodily cavities tissue. One of the essential functions of the detector 67 is to control the effect of the energy or light source on the vaginal and other bodily cavities tissue surrounding the site. In every individual case, doctors set specific characteristics of irradiation to produce the required effect. If a situation at the operation site becomes unfavorable, for example the temperature exceeds predetermined limits, the detector 67 generates a signal directed to the control unit 40, which in turn produces a correcting signal to the power unit or to the control arrangement of the system.

Electrophoresis, is known to be applicable as a therapeutic tool for certain medical conditions, often through a technique known as “iontophoresis.” Iontophoresis utilizes mild electrical currents to enhance penetration of therapeutic agents or drugs through a skin or a bodily tissue.

Referring now to FIGS. 7A, 7B and 8B illustrating further features and embodiments of the method and apparatus of the invention, which combine the therapeutic effects of laser energy radiation directed to the tissue of internal walls of bodily cavity CC with electrophoresis/iontophoresis treatment. In this embodiment of the invention, as shown in FIG. 8B a condom-type sheath 81 impregnated with a therapeutic agent or a drug medicative solution is positioned at an exterior of the protective shield 70. Multiple electrodes 77 are provided in the interior of the shield 70 and oppositely charged external electrode 79 is placed at an optimally selected location on a patient's body, a back of the patient for example (see FIG. 7B).

As illustrated in FIGS. 7A and 8B electrodes 77 are uniformly distributed across the safety shield 70 adapted for insertion into the bodily cavity CC. The electrodes 77 are electrically connected to a connector 75 located at the distal end of the shield 70 (see FIG. 4). The connector 75 interfaces with a corresponding connector 73 of the main laser device/apparatus 10, providing the delivery of electrical potential to electrodes 77. The external electrode 79 is functionally associated with control unit 40 having a microchip controller 42, facilitating control and operation of the system. The programmable logic controller (PLC) or microchip 42 controls movement of the connecting element 39 to assure that the pulsing laser energy delivered through the reflecting unit 58, does not irradiate the electrodes 77.

In this embodiment, the therapeutic agents or drugs are delivered trans-dermally into the inner wall tissues of the bodily cavity CC by applying a voltage gradient between the electrodes 77 and 79. This approach by combining the laser energy radiation with electrophoresis/iontophoresis treatment enhances the effectiveness of therapeutic agents' delivery while promoting tissue healing. As shown more specifically in FIGS. 7A and 7B, the voltage gradient is established between the electrodes 77 and 79. This voltage gradient causes ions containing medication of the sheath 81 to be drawn between the oppositely charged electrodes 77 and 79. As a result, such medication is delivered through the internal wall tissues of the body cavity CC, promoting the desired therapeutic effect. Medication containing ion molecules with embedded medicine is delivered into the internal bodily cavity in the liquid or any other state or it can be attached to the external walls of the safety shield 70. Such medication becomes active and free moving in the moist environment of the internal bodily cavities. In this embodiment by combining iontophoresis with laser energy radiation therapy, the permeability of the cavity walls is increased, facilitating improved absorption of the drugs carried by the ion delivery molecules. This enhanced drug penetration leads to a more effective therapeutic outcome.

The medical condition of inflamed tonsils is known as tonsillitis. This condition occurs when the tonsils, which are two lymphoid tissues located at the back of the throat, become swollen and irritated, usually due to an infection. Lasers are known to be used in tonsil treatments medical procedures. Laser tonsillectomy refers to a complete removal of the tonsils using a laser. On the other hand, laser tonsillotomy is a partial removal or reduction of tonsil tissue to alleviate symptoms, preserving some tonsil tissue. Tonsillitis is commonly known to be caused by viral infections, although bacterial infections can also be responsible. The primary bacterium associated with tonsillitis is Streptococcus pyogenes (group A streptococcus), which is the same bacteria that causes strep throat. Other strains of streptococcus and various bacteria can also contribute to the condition.

Laser energy is known to be effective in eliminating both bacteria and viruses. Specifically, 650-microsecond pulses of 1064 nm laser wavelength beam, when delivered at high power, can effectively target and treat an infected tissue. This treatment is both highly effective and gentle, with minimal risk of side effects. As such, by irradiating areas affected by tonsillitis with 650-microsecond 1064 nm laser energy may offer a safe and effective alternative to traditional medications or serve as an adjunct therapy to speed up recovery.

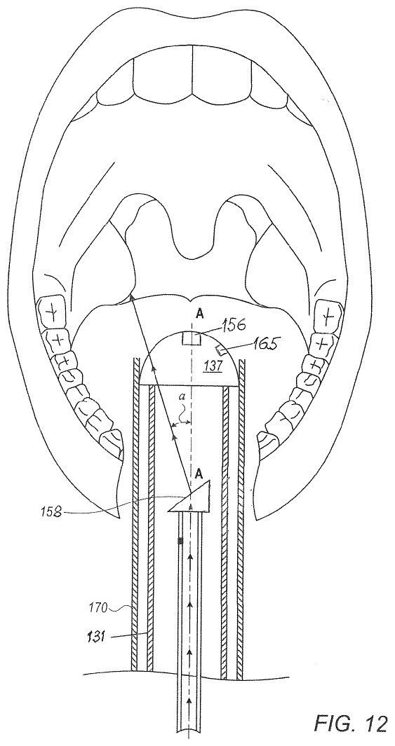

Referring now to FIG. 12 which illustrates a further embodiment of the invention provided for the convenient delivery of laser energy to a throat area of a patient. More specifically, the embodiment of FIG. 12 refers to the application of a modified laser energy dispensing arrangement previously discussed with reference to FIG. 6 to the treatment of tonsillitis. In the embodiment of FIG. 12 semi-spherical formation 137 provided at the distal region of the receiving element 131 is transparent to the laser radiation. The reflecting member or optical prism 158 is positioned to reflect the laser beam at an optimal acute angle “a” relative to the optical axis A-A of the laser. The reflected laser beam passes through the radiationally transparent semi-spherical formation 137 and the safety shield 170 to ensure precise and safe laser energy delivery to the intended target. The rotational movement of a connecting element 139 generates a rotational distribution of laser energy across a throat tissue, while the longitudinal movement of the connecting element 139 causes the laser spots to shift in a perpendicular/tangential direction within the treatment tonsils area.

In view of the combined motion of the reflective arrangement 139 including the rotational and longitude motion, the laser beam pattern effectively covers the tonsil area of the patient mouth affected by the viruses and bacteria. The laser beam reflected from the prism 158 travels passes through the radiationally transparent semi-spherical formation 137 and also passes through the safety shield 170. There are also provided sensors 165 in the formation 137 not affecting the laser beam. The imaging device 156 is also provided (see the discussion of FIG. 6 embodiment) enabling a practitioner to observe and to mark the tonsils area to be treated. The algorithm imbedded into the microchip 142 facilitates combining rotational and longitudinal movements to cover the area affected by the tonsillitis.

### TECHNOLOGICAL SUMMARY AND CONCLUSIONS

It has been discussed above that currently there are devices on the market for an external and internal treatment of female genitalia. However, these devices are separate/independent from each other, wherein the devices for internal treatment are not usable for the external application. On the other hand, the invention provides one/unitary medical device and system which provides treatment of all external and internal areas of female genital and other bodily cavities.

One of the technological approaches used in the invention is utilization of laser energy with 1064 nm wavelength. It is essential that this wavelength is absorbed by all three main components or building blocks of the skin: water, hemoglobin and melanin. This positions 1064 nm laser as a very versatile technology to treat various skin conditions. However, to achieve meaningful therapeutic effect on treatments related to feminine internal or external rejuvenation the laser energy fluence of 1064 nm laser energy must be well in excess of 20 joules per cm² on at least a 6 mm spot for deep penetration through treated tissue. Such high fluences typically can be achieved with long pulse (3 millisecond to 50 millisecond) 1064 nm Nd: YAG lasers; however, at those pulse duration 1064 nm laser treatment has a high discomfort and a risk of skin burns. This makes long pulse 1064 nm lasers particularly unacceptable for vaginal and other bodily cavities and other feminine genitalia skin areas. The apparatus of this invention proposes using 1064 nm laser with a much shorter 650 microsecond pulse duration.

Thermal relaxation time (tau(r)) is a commonly used parameter for estimating the time required for heat to conduct away from a directly heated tissue region. The invention proposes an approach based on the application of 1064 nm lasers with 650 microsecond pulse duration which is below the 800-microsecond thermal relaxation time for skin tissue. At the pulse duration below the thermal relaxation time the heat generated within the targeted skin structure stays within the target as it does not have enough time to dissipate into the surrounding skin thus minimizing the risk of overheating. Thus, the practitioner can apply optimally high fluences for a desired therapeutic effect to the female genitalia and other bodily cavities without risk of damage and burns to the skin tissue typically associated with high energy/fluence long pulse 1064 nm lasers.

Although the main object of the invention is to provide an apparatus for the treatment of the internal parts of female genitalia, in view of the flexibility of the structural composition, the apparatus of the invention also enables a practitioner to conduct an efficient treatment of the external parts of the female genitalia as well.

The invention has been primarily described with reference to apparatus and methods for the treatment of female and male bodily cavities. However, the medical treatment of both humans and animals often shares similarities, as both fields rely on analogous principles of biology and anatomy. Therefore, the application of the apparatus and methods disclosed above to the treatment of animals is also within the scope of the present invention.

What is claimed is:

1. A laser based therapy apparatus for treatment of various medical conditions associated with bodily cavities, the apparatus comprising: a laser generating device producing 1064 nm laser wavelength beam having pulse duration between 0.1 and 3 milliseconds, and more specifically having 650 microsecond pulse duration.

2. The apparatus of claim 1, wherein said medical conditions include at least vaginal and other bodily cavities infections (Bacterial vaginosis (BV), Yeast infections, etc.), hemorrhoids, vaginal laxity, stress urinary incontinence, loss of vaginal lubrication (dryness), a decrease in erotic sensation, cervical erosion, vaginal ulceration, tonsillitis and streptococcus infections.

3. The apparatus of claim 1, further comprising a detachable laser delivery device forming a unitary medical laser-based assembly, a substantially hollow external housing with a tubular receiving element accommodating the shaft, the external housing connected to the electrical motor case, the receiving element is formed having hollow interior and exterior regions.

4. The apparatus of claim 3, further comprising a disposable protective shield provided at an exterior of the receiving element, the protective shield is formed having a tubular shaped body extending between the front and rear ends; a support formation having an inverted mushroom shape formed at an exterior of the rear end of the disposable shield; an inner cavity facing the front end of the shield is formed within the support; the cavity extends between an outer area defined by an outer edge of the stopper and an inner area associated with the tubular connector; and a vacuum port or a nozzle is provided for communication of the cavity within atmosphere and to generate a vacuum for evacuation of debris developed during the treatment.

5. The apparatus of claim 1, further comprising a handpiece having multiple apertures for air to be drawn into a handpiece interior. a vacuum suction generated within the handpiece interior is extended to the inner area of a hose; upon passing through a filter the cleaned air continues its movement into the interior of the laser handpiece for cooling; so that in the apparatus of the invention air provided for cooling the regular the laser device disposed in the handpiece is also utilized to form the suction directed to remove debris produced at a treatment area and accumulated in a disposable filter.

6. The apparatus of claim 1, further comprising an electrophoresis arrangement including condom-type sheath impregnated with a therapeutic agent is positioned at an exterior of the protective shield, multiple electrodes are formed in an interior of the shield, with an oppositely charged external electrode placed at an optimally selected location on a patient's body; so that by combining the laser energy radiation with electrophoresis treatment enhances the effectiveness of therapeutic agents delivery while promoting tissue healing.

7. The apparatus of claim 1, further comprising an arrangement for delivery of laser energy to a throat area of a patient, in said arrangement a semi-spherical formation is provided at the distal region of the receiving element is transparent to the laser radiation; a reflecting member is positioned to reflect the laser beam at an optimal acute angle relative to the optical axis of the laser; the reflected laser beam passes through the radiationally transparent semi-spherical formation and the safety shield to ensure precise and safe laser energy delivery to the throat area, a rotational movement of a connecting element generates a rotational distribution of laser energy across a throat area, while a longitudinal movement of the connecting element causes the laser spots to shift in a perpendicular/tangential direction within the treatment tonsils area.

8. The apparatus of claim 7, wherein in view of a combined motion of the reflective arrangement including the rotational and longitude motion a laser beam pattern effectively covers the tonsil area of the patient; The laser beam reflected from the prism travels passes through the radiationally transparent semi-spherical formation and also passes through the safety shield.

9. The apparatus of claim 8, further comprising an imaging device for observing the tonsils area to be treated.

10. A method of treatment of various medical conditions associated with bodily cavities by means of a use of laser based therapy apparatus, said method comprising:
a step of irradiating the bodily cavities with 1064 nm laser wavelength having pulse duration between 0.1 and 3 milliseconds, and more specifically having 650 microsecond pulse duration, wherein said laser beam is produced by said therapy apparatus.

11. The method of claim 10, wherein said medical conditions include at least wherein said medical conditions include at least vaginal and other bodily cavities infections (Bacterial vaginosis (BV), Yeast infections, etc.), hemorrhoids, vaginal laxity, stress urinary incontinence, loss of vaginal lubrication (dryness), a decrease in erotic sensation, cervical erosion, vaginal ulceration, tonsillitis and streptococcus infections.

12. The method of claim 10, wherein said therapy apparatus further comprises an electrophoresis arrangement including condom-type sheath impregnated with a therapeutic agent is positioned at an exterior of the protective shield, multiple electrodes are formed in an interior of the shield, with an oppositely charged external electrode placed at an optimally selected location on a patient's body; so that by combining the laser energy radiation with electrophoresis treatment enhances the effectiveness of therapeutic agents delivery while promoting tissue healing.

13. The method of claim 1, wherein said therapy apparatus further comprises an arrangement for delivery of laser energy to a throat area of a patient, in said arrangement a semi-spherical formation is provided at the distal region of the receiving element is transparent to the laser radiation; a reflecting member is positioned to reflect the laser beam at an optimal acute angle relative to the optical axis of the laser; the reflected laser beam passes through the radiationally transparent semi-spherical formation and the safety shield to ensure precise and safe laser energy delivery to the throat area, a rotational movement of a connecting element generates a rotational distribution of laser energy across a throat area, while a longitudinal movement of the connecting element causes the laser spots to shift in a perpendicular/tangential direction within the treatment tonsils area.

14. The method of claim 13, wherein in said therapy apparatus view of a combined motion of the reflective arrangement including the rotational and longitude motion a laser beam pattern effectively covers the tonsil area of the patient; the laser beam reflected from the prism travels passes through the radiationally transparent semi-spherical formation and also passes through the safety shield.
